(19) United States
(12) Patent Application Publication
(10) Pub. No.: US 2016/0106689 A1
(43) Pub. Date: Apr. 21, 2016

(54) USE

(71) Applicant: Novabiotics Limited, Craibstone (GB)

(72) Inventor: Deborah O'NEIL, Craibstone (GB)

(73) Assignee: NOVABIOTICS LIMITED, Craibstone (GB)

(21) Appl. No.: 14/860,970

(22) Filed: Sep. 22, 2015

Related U.S. Application Data
(60) Provisional application No. 62/053,505, filed on Sep. 22, 2014.

(51) Int. Cl. A61K 31/145; A61K 45/06

(52) U.S. Cl. CPC A61K 31/145; A61K 45/06

(57) ABSTRACT

The present invention relates to compositions comprising cysteamine or a derivative thereof for use in treating infections caused by yeasts or moulds.

### CROSS-REFERENCE TO RELATED APPLICATIONS

The present application claims the benefit of U.S. provisional application No. 62/053,505 filed on Sep. 22, 2014, which is incorporated herein by reference in its entirety for all purposes.

### FIELD OF THE INVENTION

The present invention relates to the use of cystamine and derivatives thereof in the treatment and/or prevention of infection caused by bacteria and/or fungi or a disease or condition associated therewith.

### BACKGROUND TO THE INVENTION

Antibiotics are widely used, both in human/veterinary medicine and also agriculture, and this has led to an increasing problem of drug resistance to currently available antibiotics. This is particularly relevant to infectious conditions or diseases that are treated with single antibiotics (otherwise known as monotherapy). As such, there is a significant need not just for effective and safe new treatments but those that have a mode of action that minimises or negates the risk of eventual development of drug resistance in target pathogen populations and for therapies that can be used in combination with other treatments in order to minimise the opportunity for resistance and extending the utility of currently available antimicrobials.

Bacterial infections of mucous-rich environments such as the lung are common in diseases such as cystic fibrosis (CF). However, conventional antibiotics do not tend to work well in such environments and their antibacterial effectiveness is greatly diminished when used in such environments.

A microbial biofilm is a community of microbial cells embedded in an extracellular matrix of polymeric substances and adherent to a biological or a non-biotic surface. A range of microorganisms (bacteria, fungi, and/or protozoa, with associated bacteriophages and other viruses) can be found in these biofilms. Biofilms are ubiquitous in nature, are commonly found in a wide range of environments. Biofilms are being increasingly recognised by the scientific and medical community as being implicated in many infections, and especially their contribution to the recalcitrance of infection treatment.

Biofilms are etiologic agents for a number of disease states in mammals and are involved in 80% of infections in humans. Examples include skin and wound infections, middle-ear infections, gastrointestinal tract infections, peritoneal membrane infections, urogenital tract infections, oral soft tissue infections, formation of dental plaque, eye infections (including contact lens contamination), endocarditis, infections in cystic fibrosis, and infections of indwelling medical devices such as joint prostheses, dental implants, catheters and cardiac implants.

Planktonic microbes (i.e., microorganisms suspended or growing in a liquid medium) are typically used as models for antimicrobial susceptibility research as described by the Clinical and Laboratory Standards Institute (CLSI) and European Committee on Antimicrobial Susceptibility Testing (EUCAST). Microbes in biofilms are significantly more resistant to antimicrobial treatment than their planktonic counterparts. However, there is no standardized method for the study of antibiotic susceptibility of biofilm microbes.

Biofilm formation is not limited solely to the ability of microbes to attach to a surface. Microbes growing in a biofilm are able to interact more between each other than with the actual physical substratum on which the biofilm initially developed. For example, this phenomenon favours conjugative gene transfer, which occurs at a greater rate between cells in biofilms than between planktonic cells. This represents an increased opportunity for horizontal gene transfer between bacteria, and is important because this can facilitate the transfer of antibiotic resistance or virulence determinant genes from resistant to susceptible microbes. Bacteria can communicate with one another by a system known as quorum sensing, through which signalling molecules are released into the environment and their concentration can be detected by the surrounding microbes. Quorum sensing enables bacteria to co-ordinate their behaviour, thus enhancing their ability to survive. Responses to quorum sensing include adaptation to availability of nutrients, defense against other microorganisms which may compete for the same nutrients and the avoidance of toxic compounds potentially dangerous for the bacteria. It is very important for pathogenic bacteria during infection of a host (e.g. humans, other animals or plants) to co-ordinate their virulence in order to escape the immune response of the host in order to be able to establish a successful infection.

Biofilm formation plays a key role in many infectious diseases, such as cystic fibrosis and periodontitis, in bloodstream and urinary tract infections and as a consequence of the presence of indwelling medical devices. The suggested mechanisms by which biofilm-associated microorganisms elicit diseases in their host include the following: (i) delayed penetration of the antimicrobial agent through the biofilm matrix, (ii) detachment of cells or cell aggregates from indwelling medical device biofilms, (iii) production of endotoxins, (iv) resistance to the host immune system, (v) provision of a niche for the generation of resistant organisms through horizontal gene transfer of antimicrobial resistance &/or virulence determinant genes, and (vi) altered growth rate (.i.e. metabolic dormancy) (Donlan and Costerton, Clin Microbiol Rev 15: 167-193, 2002; Parsek and Singh, Annu Rev Microbiol 57: 677-701, 2003; Costerton J W, Resistance of biofilms to stress. In ‘The biofilm primer’. (Springer Berlin Heidelberg). pp. 56-64.2007).

Recent experimental evidence has indicated the existence within biofilms of a small sub-population of specialized non-metabolising persister cells (dormant cells). It is thought that these cells may be responsible for the high resistance/tolerance of biofilm to antimicrobial agents. Multi-drug-tolerant persister cells are present in both planktonic and biofilm populations and it appears that yeasts and bacteria have evolved analogous strategies that assign the function of survival to this sub-population. The protection offered by the polymeric matrix allows persister cells to evade elimination and serve as a source for re-population. There is evidence that persisters may be largely responsible for the multi-drug tolerance of microbial biofilms (LaFleur et al., Antimicrob Agents Chemother. 50: 3839-46, 2006; Lewis, Nature Reviews Microbiology 5, 48-56 2007).

There remains a need for better therapies for treating and preventing bacterial infections, in particular those associated with mucous-rich environments such as the CF lung. In addition there remains a need to limit the amount or doses of antibiotics used with the introduction of novel, replacement therapies or adjunct treatments that can improve the effectiveness of currently available treatments in the treatment or prevention of bacterial infections, in particular in a biofilm setting.

The frequency of invasive fungal infections has also continued to increase over the past two decades, both in the general population and in immunosuppressed patients with the vast majority of infections caused by Aspergillus and Candida species (Pasqualotto, A. C., and Denning, D. W. (2005) Diagnosis of Invasive Fungal Infections—Current Limitations of Classical and New Diagnostic Methods. Euro Oncol Rev). These infections carry high mortality rates and place significant burdens on health care systems. There remains an urgent need for more effective and safe therapeutic agents to treat and prevent infections by yeasts and mould (including e.g. Candida spp. or Aspergillus spp.).

### STATEMENTS OF THE INVENTION

According to a first aspect of the invention, there is provided cystamine for use in the treatment of a bacterial, fungal or parasitic infection or disease or condition associated therewith.

The inventors have surprisingly found that an effective amount cystamine has good antibacterial and/or antifungal and/or mucolytic properties

Cystamine derived from cysteamine administered in vivo surprisingly has good antibacterial, antifungal and muclytic properties without causing toxicity issues.

Suitably, the bacterial or fungal infection may be a biofilm infection.

Suitably, the infection, or disease or condition associated therewith, may be selected from the group consisting of respiratory infections, infections in cystic fibrosis, skin and wound infections, middle-ear infections, gastrointestinal tract infections, peritoneal membrane infections, urogenital tract infections, oral soft tissue infections, formation of dental plaque, eye infections, endocarditis and infections of indwelling medical devices.

Suitably, the infection may be a bacterial infection, such as a bacterial infection by one or more of the following bacterial species selected from the group consisting of: Staphylococcus spp., e.g. Pseudomonas spp; Staphylococcus aureus, Staphylococcus epidermidis; Enterococcus spp., e.g. Enterococcus faecalis; Streptococcus pyogenes; Listeria spp.; Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli; Haemophilus spp., e.g. Haemophilus influenzae; Chlamydia spp., e.g. Chlamydia trachomatis, Chlamydia pneumoniae, Chlamydia psittaci; Francisella tularensis; Bacillus spp., e.g. Bacillus anthracis; Clostridia spp., e.g. Clostridium botulinum; Yersinia spp., e.g. Yersinia pestis; Treponema spp.; Burkholderia spp.; e.g. Burkholderia mallei and B pseudomallei.

Suitably, the infection may be a fungal infection, such as a fungal infection by one or more of the following species selected from the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

Suitably, the infection may be by Candida spp., (e.g. C. albicans) or Aspergillus fumigatus.

Suitably, the infection may be a parasitic infection, such as an infection by Trypanosoma spp. (Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp., Crytosporidium spp., Isospora spp., Balantidium spp., Loa Loa, Ascaris lumbricoides, Dirofilaria immitis, Toxoplasma ssp., e.g Toxoplasma gondii.

Suitably, the disease associated with the infection may be selected from the group consisting of: candidiasis (including OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis.

Suitably, cystamine may be used in a pharmaceutical composition comprising a pharmaceutically acceptable carrier, excipient or diluent

Suitably, cystamine for use in accordance with the present invention may be used in combination with an antibiotic. Preferably, the antibiotic is selected from one or more of the group consisting of: Tobramycin, Colistin, Azithromycin, Gentamicin or Ciprofloxacin.

Suitably, cystamine for use in accordance with the present invention may be used in combination with a peptide comprising from 3 to 50 alpha, D and/or L amino acids wherein the amino acids are predominantly arginine and wherein the peptide, optionally further comprises a modification which is selected from one or more of the group consisting of:
1) Incorporation of a histidine tag;
2) lipidation; and
3) pegylation.

Suitably, cystamine for use in accordance with the present invention may be used in combination with a peptide comprising from 3-500 amino acids wherein the amino acids are predominantly arginine.

In a further aspect, the present invention relate to a method of treating a bacterial or fungal infection or disease or condition therewith associated in a patient comprising administering an effective amount of cystamine to the patient.

Suitably, the bacterial or fungal infection is a biofilm infection.

Suitably, the infection, or disease or condition associated therewith, may be selected from the group consisting of respiratory infections, infections associated with cystic fibrosis, skin and wound infections, middle-ear infections, gastrointestinal tract infections, peritoneal membrane infections, urogenital tract infections, oral soft tissue infections, formation of dental plaque, eye infections, endocarditis, and infections of indwelling medical devices.

Suitably, the infection may be a bacterial infection, such as a bacterial infection by one or more of the following bacterial species selected from the group consisting of: Staphylococcus spp., e.g. Pseudomonas spp; Staphylococcus aureus, Staphylococcus epidermidis; Enterococcus spp., e.g. Enterococcus faecalis; Streptococcus pyogenes; Listeria spp.; Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli; Haemophilus spp., e.g. Haemophilus influenzae; Chlamydia spp., e.g. Chlamydia trachomatis, Chlamydia pneumoniae, Chlamydia psittaci; Francisella tularensis; Bacillus spp., e.g. Bacillus anthracis; Clostridia spp., e.g. Clostridium botulinum; Yersinia spp., e.g. Yersinia pestis; Treponema spp.; Burkholderia spp.; e.g. Burkholderia mallei and B pseudomallei.

Suitably, the infection may be a fungal infection, such as a fungal infection by one or more of the following species selected from the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

Suitably, the infection may be a parasitic infection, such as an infection by Trypanosoma spp. (Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp., Crytosporidium spp., Isospora spp., Balantidium spp., Loa Loa, Ascaris lumbricoides, Dirofilaria immitis, Toxoplasma ssp., e.g Toxoplasma gondii.

Suitably, the infection may be by Candida spp., (e.g. C. albicans) or Aspergillus fumigatus.

Suitably, the infection may be selected from the group consisting of: candidiasis (including OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis.

Suitably, cystamine may be used in combination with an antibiotic. Preferably, the antibiotic is selected from one or more of the group consisting of: Tobramycin, Colistin, Gentamicin or Ciprofloxacin.

Suitably, cystamine may be used in combination with a peptide comprising from 3 to 50 alpha, D and/or L amino acids wherein the amino acids are predominantly arginine and wherein the peptide, optionally further comprises a modification which is selected from one or more of the group consisting of:
1) Incorporation of a histidine tag;
2) lipidation; and
3) pegylation.

Suitably, cystamine may be used in combination with a peptide comprising from 3-500 amino acids wherein the amino acids are predominantly arginine.

In another aspect of the present invention, there is provided a method of preventing biofilm formation in an environment comprising the step of administering an effective amount of cystamine to the environment. Suitably, the environment may comprise a biofilm forming microorganism selected from bacteria and fungi.

Suitably, the microorganism may be a bacterium such as a bacterium selected from the group consisting of may include Pseudomonas spp., Staphylococcus spp., Haemophilus spp., Burkholderia spp., Streptococcus spp., Propionibacterium spp. Preferably, the bacterium is selected from Pseudomonas spp., and Staphylococcus spp. Preferably, the bacterium is Pseudomonas aeruginosa, Staphylococcus aureus or Staphylococcus epidermidis.

Suitably, the environment may be the mouth.

Suitably, the microbial infection may be a systemic infection. Preferably, the systemic infection is a mucosal infection.

Suitably, the mucosal infection may be a gastrointestinal, urogenital or respiratory infection.

Suitably, the mucosal infection may be cystic fibrosis.

In a further aspect, the present invention relates to the use of cystamine as an antibiofilm agent. The term “antibiofilm agent” is used herein to describe an agent that is capable of destroying or inhibiting the growth of a microbial biofilm. The antibiofilm agent may be capable of disrupting the structure of the biofilm, for example the extracellular mucous matrix, or may be capable of destroying or inhibiting the growth of microbial cells within the biofilm.

### DETAILED DESCRIPTION

The present invention provides cystamine for use in the treatment of a bacterial or fungal infection or disease or condition associated therewith as well as method of treatment of a bacterial or fungal infection or disease or condition associated therewith.

Typically the microorganism is a bacterium for example a Gram-positive or Gram-negative bacterium. A bacterial pathogen may be derived from a bacterial species selected from the group consisting of: Staphylococcus spp., e.g. Staphylococcus aureus, Staphylococcus epidermidis; Enterococcus spp., e.g. Enterococcus faecalis; Streptococcus pyogenes; Listeria spp.; Pseudomonas spp.; Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli; Haemophilus spp., e.g. Haemophilus influenzae; Chlamydia spp., e.g. Chlamydia trachomatis, Chlamydia pneumoniae, Chlamydia psittaci; Francisella fularensis; Bacillus spp., e.g. Bacillus anthracis; Clostridia spp., e.g. Clostridium botulinum; Yersinia spp., e.g. Yersinia pestis; Treponema spp.; Burkholderia spp.; e.g. Burkholderia mallei and B pseudomallei.

In particular the bacterium may include Pseudomonas spp., for example Pseudomonas aeruginosa; Staphylococcus spp., for example Staphylococcus aureus and Staphylococcus epidermidis; Haemophilus spp., for example Haemophilus influenza; Burkholderia spp., for example Burkholderia cepacia; Streptococcus spp., Propionibacterium spp., for example Propionibacterium acnes. Preferably the bacterium is selected from Pseudomonas spp., for example Pseudomonas aeruginosa and Staphylococcus spp., for example Staphylococcus aureus and Staphylococcus epidermidis.

Suitably, the infection may be a fungal infection, such as a fungal infection by one or more of the following species selected from the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

Suitably, the infection may be a parasitic infection, such as an infection by Trypanosoma spp. (Trypanosoma cruzi, Trypansosoma brucei), Leishmania spp., Giardia spp., Trichomonas spp., Entamoeba spp., Naegleria spp., Acanthamoeba spp., Schistosoma spp., Plasmodium spp., Crytosporidium spp., Isospora spp., Balantidium spp., Loa Loa, Ascaris lumbricoides, Dirofilaria immitis, Toxoplasma ssp., e.g Toxoplasma gondii.

The present invention also provides a method of preventing biofilm formation or disrupting existing biofilms in an environment comprising the step of administering an effective amount of cystamine to the environment.

Without wishing to be bound by theory, it is believed that cystamine demonstrates antibacterial activity against, inter alia, the persister cells present in the biofilms, which is an essential step towards the eradication of biofilms.

Suitably, cystamine may be used as a dispersant. The term “dispersant” refers to an agent capable of dispersing the particles of a biofilm or mucous. In particular, the dispersant may promote the dispersion of slime produced by microbes such as bacteria, mucous which forms part of the biofilm for example mucous produced by the cells to which the biofilm microbes adheres, and biofilm microbes such as bacteria.

In a further aspect of the invention there is provided a substrate to which cystamine is applied or attached. Preferably, the substrate is suitable for application to wounds or delivery to wound sites. Preferably, the substrate allows for the transfer of the active agents from the substrate to a wound bed to achieve their antibiofilm effect. The substrate may be a dressing, for example, wound dressing. The dressing may comprise a fabric material or it may be a collagen-like material. The substrate may be in any suitable form for application to a wound, typically the substrate may be in the form of a hydrogel, colloid, ointment, cream, gel, foam or spray.

Cystamine may also find application as/in a disinfectant or biocide. In this context, the peptide or pharmaceutical compositions of the invention may be applied, either alone or in combination with other disinfecting agents, to a surface to be treated. As used herein a “surface to be treated” may be a substrate as defined herein and may include medical devices and indwelling devices, e.g. stents, catheters, peritoneal dialysis tubing, draining devices, joint prostheses, dental implants and the like.

### Optional Modified Peptide

Cystamine may optionally be used in combination with a modified peptide comprising from 3 to 50 D and/or L amino acids wherein the amino acids are predominantly arginine and wherein the peptide comprises a modification which is selected from one or more of the group consisting of:
1) Incorporation of a histidine tag;
2) lipidation; and
3) pegylation

### Incorporation of a Histidine Tag

In one aspect, the modified peptide when used preferably comprises a histidine tag at either the N terminus or C terminus. Advantageously, the presence of a histidine may enhance the effectiveness of the peptide against fungal infections such as Candida. This is extremely unexpected given that the cationic charge may not be significantly changed at such a pH range when compared to an equivalent peptide without the presence of a histidine tag.

Suitably, the histidine tag may comprise at least two histidine residues. Preferably, the number of histidine residues may be up to 10. For example the histidine tag may consist of 1 to 10 histidine residues, preferably 2 to 6. In one embodiment, the histidine tag may consist of two histidine residues

Advantageously, the presence of a histidine tag may be particularly useful for treating fungal infections of the mouth such as oropharyngeal candidiasis.

The oral cavity has a pH between 5.5 and 7 in disease states whereas the normal pH of the mouth of a healthy oral cavity is around pH 7 when not feeding. However, pH influences the charge of AMPs. Furthermore, secreted saliva also contains proteases that aid the breakdown of peptides.

The present inventors have surprisingly found that peptides modified to comprise a histidine tag are particularly adept at overcoming the pH and protease challenges associated which oral administration.

Accordingly, modified peptides comprising a histidine tag may be comprised in pharmaceutical formulations adapted for oral administration.

Suitably, the peptide used may comprises a histidine tag when the route of administration or intended route of administration is oral administration.

Preferably, the pH of the pharmaceutical compositions of the present invention is in the region of pH 5.5 to 6.5.

### Lipidation

In one aspect, the modified peptides when used in combination with cystamine are lipidated. For example, a lipid may be conjugated to a peptide comprising from 3 to 50 D and/or L amino acids wherein the amino acids are predominantly arginine.

The present invention has surprisingly found that lipidation of the peptides can advantageously broaden the spectrum of activity of the peptides against microbes and/or enhance the activity of the peptides against some microbial infections.

Suitably, lipidated peptides of the present invention may be used in the treatment or prevention of yeast and mould infections (preferably such as Candida and/or Aspergillus infections, preferably Aspergillus infections). It has been surprisingly found that lipidation of the peptides claimed can confer potent activity on such lipidated peptides.

Accordingly, the modified peptide may comprise a lipid which may be at either the C terminus, N terminus or flanked with amino acid residues.

Suitably the peptide may comprise a C₃ to C₂₀ fatty acid, preferably a C₄ to C₁₄ fatty acid, preferably a C₈ to C₁₄ fatty acid, preferably a C₁₂ fatty acid.

Suitably the modified peptide may comprise 3 to 50 amino acids and a C₃ to C₂₀ fatty acid, preferably a C₄ to C₁₄ fatty acid, preferably a C₈ to C₁₄ fatty acid, preferably a C₁₂ fatty acid. Preferably, the modified peptides of the present invention may comprise 6 to 50 amino acids and a C₃ to C₂₀ fatty acid, preferably a C₄ to C₁₄ fatty acid, preferably a C₈ to C₁₄ fatty acid, preferably a C₁₂ fatty acid.

In one aspect, the fatty acid may be flanked on either side by amino acid residues. It has surprisingly been found that the flanking of the fatty acid can lead to a reduction in haemolytic activity.

In another aspect, the fatty acid may be located on the terminus of the peptide. It has surprisingly been found that this may increase the antimicrobial effects of the peptide in terms of lower MIC.

In one preferable embodiment the fatty acid is a C₁₂ fatty acid. Advantageously, this length of fatty acids exhibits both good antimicrobial effects and additionally has low cytotoxicity and haemolytic activity.

### PEGylated Peptides

In one aspect the modified peptide is a PEGylated peptide.

Advantageously, such PEGylated peptides have enhanced stability whilst still providing antimicrobial effects. Suitably, the size of the PEG component may be approx. 300 Da to approx. 40 KDa

### Amino Acid Residues

The peptide may comprise from 3 to 50 (preferably contiguous) amino acids.

Suitably the peptide may comprise at least 3 or at least 4 or at least 5 or at least 6 or at least 7 or at least 8 or at least 9 or at least 10 or at least 12 or at least 15 or at least 20 or at least 25 or at least 30 or at least 35 or at least 40 or at least 45 amino acids.

Suitably, the peptide may comprise less than 50 or less than 45 or less than 40 or less than 35 or less than 30 or less than 25 or less than 0 or less than 15 amino acids.

In one aspect the number of amino acid residues referred to in the ranges above does not include the histidine tag residues. Thus, in one aspect, histidine residues at either end of the peptide are discounted when determining the numbering of amino acids in the modified peptide. In another aspect, all amino acid residues are counted including those making up a histidine tag.

In a preferred aspect of the invention the peptide comprises 3 to 20 (preferably contiguous) amino acids, for example 3 to 16 amino acids. Preferably still the peptide comprises 5 to 14 amino acids. In some aspects, the peptide may comprise 12 (preferably contiguous) amino acids.

As known to the skilled person, amino acids can be placed into different classes depending primarily upon the chemical and physical properties of the amino acid side chain. For example, some amino acids are generally considered to be hydrophilic or polar amino acids and others are considered to be hydrophobic or non-polar amino acids. Hydrophobic amino acid may be selected from the group of hydrophobic amino acids consisting of glycine, leucine, phenylalanine, proline, alanine, tryptophan, valine, isoleucine, methionine, tyrosine and threonine; cationic amino acids may be selected from the group consisting of ornithine, histidine, arginine and lysine. As used herein, the terms “hydrophobic” and “cationic” may refer to amino acids having a hydrophobicity that is greater than or equal to −1.10 and/or a net charge that is greater than or equal to 0 as described in Fauchere and Pliska Eur. J. Med Chem. 10:39 (1983). A hydrophobic or non-polar amino acid may also refer to an amino acid having a side chain that is uncharged at physiological pH, is not polar and that is generally repelled by aqueous solution. The amino acids may be naturally occurring or synthetic.

Suitably, the arginine residue is the predominant amino acid in the peptide. Suitably, at least 50% of the amino acid residues are arginine residues, preferably at least 60% or at least 70% or at least 80% of the amino acids in the peptide are arginine. Preferably, at least 90% are arginine residues. In some embodiments all the amino acids in the peptide are arginine residues (optionally with the exception of a histidine tag).

Suitably, the peptide may comprise amino acids other than arginine is non-predominant amounts. For example, histidine, ornithine and lysine could be used.

Suitably, 3 to 50 (preferably contiguous) D and/or L amino acids consist of arginine or a combination of arginine and lysine residues except for 0, 1, or 2 substitutions to an amino acid residues other than arginine or lysine. Preferably, such substitutions (if present) are with another cationic amino acids selected from the group consisting of histidine, ornithine and lysine. Preferably the substations are with lysine.

Suitably, the peptide may be substituted with 0, 1, 2, 3, 4, 5, 6, 7 or 8 substitutions provided that the arginine make up at least 60%, preferably at least 75% of the peptide. Preferably, the amino acids are L-amino acids.

In a preferred aspect of the invention, at least 90%, for example at least 95% such as 97-99% or even 100%, of the amino acids in the peptide are L-amino acids.

The invention also includes known isomers (structural, stereo-, conformational & configurational), peptidomimetics, structural analogues of the above amino acids, and those modified either naturally (e.g. post-translational modification) or chemically, including, but not exclusively, phosphorylation, glycosylation, sulfonylation and/or hydroxylation.

In general, the peptide does not include the amino acids aspartic acid, glutamic acid, asparagine, glutamine or serine, but certain peptides of the invention may have activity even though these amino acids are present.

One or more of the residues of the peptide can be exchanged for another to alter, enhance or preserve the biological activity of the peptide. Such a variant can have, for example, at least about 10% of the biological activity of the corresponding non-variant peptide. Conservative amino acids are often utilised, i.e. substitutions of amino acids with similar chemical and physical properties as described above. Hence, for example, conservative amino acid substitutions may involve exchanging lysine for arginine, ornithine or histidine; or exchanging arginine for lysine or isoleucine, ornithine for histidine; or exchanging one hydrophobic amino acid for another. After the substitutions are introduced, the variants are screened for biological activity.

### Peptides

The term “peptide” as used herein means, in general terms, a plurality of amino acid residues joined together by peptide bonds. It is used interchangeably and means the same as polypeptide and protein.

The term “modified peptide” refers to a peptide comprising 3 to 50 amino acid residues predominantly arginine further comprising: a histidine tag; and/or a fatty acid and/or a pegylated peptide. Suitably, the modified peptides of the present invention, may be linear peptides.

Preferably, the modified peptide may consist of:
1) 3 to 50 amino acid residues predominantly arginine and a histidine tag;
2) 3 to 50 amino acid residues predominantly arginine and one or more fatty acids;
3) 3 to 50 amino acid residues predominantly arginine, a histidine tag and one or more fatty acids;
4) a PEGylated peptide of 3 to 50 amino acid residues predominantly arginine and a histidine tag;
5) a PEGylated peptide of 3 to 50 amino acid residues predominantly arginine and one or more fatty acids; or
6) a PEGylated peptide of 3 to 50 amino acid residues predominantly arginine, a histidine tag and one or more fatty acids.

The peptides may generally be synthetic peptides. The peptides may be isolated, purified peptides or variants thereof, which can be synthesised in vitro, for example, by a solid phase peptide synthetic method, by enzyme catalysed peptide synthesis or with the aid of recombinant DNA technology.

To identify active peptides that have little or no undesired toxicity for mammalian cells, individual peptides, or libraries of peptides, can be made and the individual peptides or peptides from those libraries can be screened for antimicrobial activity and toxicity, including, but not limited to, antifungal, antibacterial, antiviral, antiprotozoal, anti-parasitic activity and toxicity.

The peptides of the invention can exist in different forms, such as free acids, free bases, esters and other prodrugs, salts and tautomers, for example, and the invention includes all variant forms of the compounds.

Thus, the invention encompasses the salt or pro-drug of a peptide or peptide variant of the invention.

### Administration

By an “effective” amount or “therapeutically effective amount” is meant an amount of one or more active substances which, within the scope of sound medical judgment, is sufficient to provide a desired effect without excessive toxicity, irritation, allergic response, or other problem or complication, commensurate with a reasonable benefit/risk ratio.

The composition of the invention may be administered in the form of a pharmaceutically acceptable salt. The pharmaceutically acceptable salts of the present invention can be synthesized from the parent peptide which contains a basic or acidic moiety by conventional chemical methods. Generally, such salts can be prepared by reacting the free acid or base forms of the peptide with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the composition of the invention wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the invention may be prepared in the usual manner by contacting the peptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

The invention includes prodrugs for the active pharmaceutical species of the described peptide, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

A further aspect of the invention provides a pharmaceutical composition comprising a pharmaceutically effective amount of cystamine. When a modified peptide is used this may be added simultaneously or may be administered separately.

The composition may also include a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

The bacterial or fungal infection may be a systemic, respiratory, topical, subcutaneous, cutaneous or mucosal infection. Preferably, the fungal infection may be a systemic, respiratory or mucosal infection.

The present invention has surprisingly found that cystamine is a potent antibacterial and antifungal for a wide variety of pathogenic bacteria, yeast and moulds. However, cystamine may also be useful in the treatment of other conditions including, but not limited to, conditions associated with mucosal infections, for example, cystic fibrosis, gastrointestinal, urogenital, urinary (e.g kidney infection or cystitis) or respiratory infections.

The term “treatment” relates to the effects of the peptides described herein that in imparting a benefit to patients afflicted with an (infectious) disease, including an improvement in the condition of the patient or delay in disease progression.

In a further aspect, the invention provides a method of treating or preventing a microbial infection in a subject comprising administering to said subject a therapeutically effective amount of cystamine.

In a preferred method of the invention, the microbial infection is a bacterial or fungal infection. In the method of the invention the peptide is preferably administered orally.

Mammals, birds and other animals may be treated by the peptides, compositions or methods described herein. Such mammals and birds include humans, dogs, cats and livestock, such as horses, cattle, sheep, goats, chickens and turkeys and the like. Moreover, plants may also be treated by the peptides, compositions or methods of the invention.

Where the subject is an animal, the method of the invention may be applied to nail-like features, including, but not exclusive to, hooves, claws and trotters.

To achieve the desired effect(s), the composition, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight or at least about 1 mg/kg to about 20 mg/kg of body weight, although other dosages may provide beneficial results. The amount administered will vary depending on various factors including, but not limited to, the peptide chosen and its clinical effects, the disease, the weight, the physical condition, the health, the age of the mammal, whether prevention or treatment is to be achieved, and if the peptide is chemically modified.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the peptides of the invention may be essentially continuous over a pre-selected period of time or may be in a series of spaced doses. Both local and systemic administration is contemplated.

To prepare the composition, cystamine is synthesized or otherwise obtained, purified as necessary or desired, and then lyophilized and stabilized. The composition can then be adjusted to the appropriate concentration and optionally combined with other agents. The absolute weight of a given peptide included in a unit dose can vary widely. For example, about 0.01 mg to about 2 g or about 0.01 mg to about 500 mg, of at least one peptide of the invention, or a plurality of peptides specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Thus, one or more suitable unit dosage forms comprising the therapeutic composition of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic peptides may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well-known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic compositions of the invention are prepared for oral administration, they are generally combined with a pharmaceutically acceptable carrier, diluent or excipient to form a pharmaceutical formulation, or unit dosage form. For oral administration, the peptides may be present as a powder, a granular formation, a solution, a suspension, an emulsion or in a natural or synthetic polymer or resin for ingestion of the active ingredients from a chewing gum. The active ingredients may also be presented as a bolus, electuary or paste. Orally administered therapeutic compositions of the invention can also be formulated for sustained release, e.g., cystamine can be coated, micro-encapsulated, or otherwise placed within a sustained delivery device. The total active ingredients in such formulations comprise from 0.1% to 99.9% by weight of the formulation.

Pharmaceutical formulations containing the therapeutic composition of the invention can be prepared by procedures known in the art using well-known and readily available ingredients. For example, the peptide can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatine, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the composition of the invention can include buffering agents such as calcium carbonate, magnesium oxide and magnesium carbonate. Suitable buffering agents may also include acetic acid in a salt, citric acid in a salt, boric acid in a salt and phosphoric acid in a salt. Caplets and tablets can also include inactive ingredients such as cellulose, pregelatinized starch, silicon dioxide, hydroxyl propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatine capsules containing at least one peptide of the invention can contain inactive ingredients such as gelatine, microcrystalline cellulose, sodium lauryl sulphate, starch, talc, and titanium dioxide, and the like, as well as liquid vehicles such as polyethylene glycols (PEGs) and vegetable oil. Moreover, enteric-coated caplets or tablets containing one or more peptides of the invention are designed to resist disintegration in the stomach and dissolve in the more neutral to alkaline environment of the duodenum.

The therapeutic composition of the invention can also be formulated as elixirs or solutions for convenient oral administration or as solutions appropriate for parenteral administration, for instance by intramuscular, subcutaneous, intraperitoneal or intravenous routes. The pharmaceutical formulations of the invention can also take the form of an aqueous or anhydrous solution or dispersion, or alternatively the form of an emulsion or suspension or salve.

Thus, the therapeutic compositions may be formulated for parenteral administration (e.g. by injection, for example, bolus injection or continuous infusion) and may be presented in unit dose form in ampules, pre-filled syringes, small volume infusion containers or in multi-dose containers. The active ingredients may form suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution for constitution with a suitable vehicle, e.g. sterile, pyrogen-free water before use.

These formulations can contain pharmaceutically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils and polysiloxanes.

Solvents or diluents comprising the peptides of the invention may include acid solutions, dimethylsulphone, N-(2-mercaptopropionyl) glycine, 2-n-nonyl-1,3-dioxolane and ethyl alcohol. Preferably the solvent/diluent is an acidic solvent, for example, acetic acid, citric acid, boric acid, lactic acid, propionic acid, phosphoric acid, benzoic acid, butyric acid, malic acid, malonic acid, oxalic acid, succinic acid or tartaric acid.

Also contemplated are combination products that include one or more peptides of the present invention and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that the peptides might be combined with topical antifungal agents such as ciclopirox olamine, haloprogin, tolnaftate, undecylenate, topical nysatin, amorolfine, butenafine, naftifine, terbinafine, and other topical agents.

Additionally, the compositions may be formulated as sustained release dosage forms and the like. The formulations can be so constituted that they release the active peptide, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

For topical administration, the active agents may be formulated as is known in the art for direct application to a target area. Forms chiefly conditioned for topical application take the form, for example, of creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration. Alternatively, the composition can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized The backing layer can be any appropriate thickness that will provide the desired protective and support functions. A suitable thickness will generally be from about 10 microns to about 200 microns.

Topical administration may be in the form of a nail coating or lacquer. For example, the antifungal peptides can be formulated in a solution for topical administration that contains ethyl acetate (NF), isopropyl alcohol (USP), and butyl monoester of poly[methylvinyl ether/maleic acid] in isopropyl alcohol.

Pharmaceutical formulations for topical administration may comprise, for example, a physiologically acceptable buffered saline solution containing between about 0.001 mg/ml and about 100 mg/ml, for example between 0.1 mg/ml and 10 mg/ml, of one or more of the peptides of the present invention specific for the indication or disease to be treated. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides can also be delivered via iontophoresis, e.g., as disclosed in U.S. Pat. No. 4,140,122; 4,383,529; or 4,051,842. The percentage by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

The therapeutic peptide may further be formulated for topical administration in the mouth or throat. For example, the active ingredients may be formulated as a lozenge further comprising a flavoured base, usually sucrose and acacia or tragacanth; pastilles comprising the composition in an inert base such as gelatine and glycerine or sucrose and acacia; and mouthwashes comprising the composition of the present invention in a suitable liquid carrier. Alternatively, the active ingredients may be formulated as a film strip or buccal tablet, which may or may not be dissolvable.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The compositions of the invention can also be administered to the respiratory tract. For administration by inhalation or insufflation, the composition may take the form of a dry powder, for example, a powder mix of the therapeutic agent and a suitable powder base such as lactose or starch. Therapeutic peptides of the present invention can also be administered in an aqueous solution when administered in an aerosol or inhaled form. Thus, other aerosol pharmaceutical formulations may comprise, for example, a physiologically acceptable buffered saline solution containing between about 0.001 mg/ml and about 100 mg/ml for example between 0.1 and 100 mg/ml, such as 0.5-50 mg/ml, 0.5-20 mg/ml, 0.5-10 mg/ml, 0.5-5 mg/ml or 1-5 mg/ml of one or more of the peptides of the present invention specific for the indication or disease to be treated.

### Antibiotic Agent

Cystamine may optionally be used in combination with an antibiotic. The term “antibiotic” is used to refer to antibacterial agents that may be derived from bacterial sources. Antibiotic agents may be bactericidal and/or bacteriostatic.

Generally the antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracyclins. Alternatively or additionally the antibiotic agent may be effective against mycobacteria.

According to one embodiment, the antibiotic agent may be an aminoglycoside such as Amikacin, Gentamicin, Kanamycin, Neomycin, Netilmicin, Tobramycin or Paromomycin. Preferably, tobramycin.

According to one embodiment, the antibiotic agent may be an http://en.wikipedia.org/wiki/Ansamycin such as Geldanamycin and Herbimycin

Alternatively the antibiotic agent may be a carbacephem such as Loracarbef.

According to a further embodiment, the antibiotic agent is a carbapenem such as Ertapenem, Doripenem, Imipenem/Cilastatin or Meropenem.

Alternatively the antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively the antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole.

The antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

Alternatively the antibiotic agent may be a monobactams such as Aztreonam, or a nitrofuran such as Furazolidone or Nitrofurantoin.

The antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

The antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

The antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

According to one embodiment, the antibiotic agent may be a polypeptide such as Bacitracin, Colistin and Polymyxin B.

Alternatively, the antibiotic agent may be a tetracycline such as Demeclocycline, Doxycycline, Minocycline and Oxytetracycline

Alternatively or additionally the antibiotic agent may be effective against mycobacteria.

In particular the antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin.

Generally the antibiotic agent is active in the treatment or prophylaxis of infections caused by gram-negative or gram-positive bacteria, such as Escherichia coli and Klebsiella particularly Pseudomonas aeruginosa.

The ratio of cystamine to antibiotic in the products of the invention may be from 1:10 to 10:1; generally at least 2:1 for example at least 3:1 or 4:1. Alternatively, the ratio of the antibiotic to cystamine in the products of the invention may be from 1:100 1:2000, for example from 1:500 to 1:1000. According to one embodiment, the ratio of the antibiotic agent to cystamine is approximately 1:1. Preferably the antibiotic is a non-peptide antibiotic and the ratio from 2:1 up to 4:1 to cystamine. According to a further embodiment the ratio may be approximately 1:1.

The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### EXAMPLES

The following Example illustrates the invention.

### Example 1

### Antimicrobial Activity Date of Cystamine Against P. aeruginosa

The stability of the thiol cysteamine and comparing its antibacterial activity with the disulphide cystamine was undertaken.

The MIC₁₀₀ using cysteamine, oxidised cysteamine and cystamine against the type strain of Pseudomonas aeruginosa (PAO1) was determined. Cysteamine and ‘oxidised’ cysteamine (containing some cystamine) were consistently equivalent at either 250 μg/ml or (as shown in the summary table below) 500 μg/ml. We oxidised the cysteamine by leaving the powder exposed to the air (in a sterile environment) for 5 days—during which time it changes in appearance (turning from an off white crystalline morphology to more of a white powder). It often also accrues water as it is hygroscopic and can also react with air to produce water. Despite these changes, as demonstrated, antibacterial activity is unaffected by this time point compared with fresh cysteamine (kept in a sealed bottle away from light at 4 degrees and in the presence of desiccant). The disulphide, cystamine (one of the potential products of cysteamine oxidation) was also tested and shown to be antibacterial.

TABLE 1
the summary table below shows the MIC₁₀₀ from the
latest set of experiments which used three replicates and
was repeated three times.

| | | 5-day oxidised | Cystamine |
|---|---|---|---|
| | Cysteamine | cysteamine | HCl |
| MIC₁₀₀ against PAO1 (μg/ml) | 500 | 500 | 1000 |
| in MHB | | | |
| MIC₁₀₀ against PAO1 (μg/ml) | 500 | 500 | 1000 |
| in CA-MHB | | | |

1.-39. (canceled)

40. A method of treating a bacterial or fungal infection or disease or condition therewith associated in a patient comprising administering an effective amount of cystamine to the patient.

41. The method of claim 40 wherein the bacterial or fungal infection is a biofilm infection.

42. The method of claim 40, wherein the infection, or disease or condition associated therewith, is selected from the group consisting of skin and wound infections, middle-ear infections, gastrointestinal tract infections, peritoneal membrane infections, urogenital tract infections, oral soft tissue infections, formation of dental plaque, eye infections, endocarditis, infections in cystic fibrosis, and infections of indwelling medical devices.

43. The method of claim 40, wherein the infection is a bacterial infection.

44. The method of claim 43, wherein the bacterial infection is by one or more of the following bacterial species selected from the group consisting of: Staphylococcus spp., e.g. Pseudomonas spp; Staphylococcus aureus, Staphylococcus epidermidis; Enterococcus spp., e.g. Enterococcus faecalis; Streptococcus pyogenes; Listeria spp.; Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli; Haemophilus spp., e.g. Haemophilus influenzae; Chlamydia spp., e.g. Chlamydia trachomatis, Chlamydia pneumoniae, Chlamydia psittaci; Francisella tularensis; Bacillus spp., e.g. Bacillus anthracis; Clostridia spp., e.g. Clostridium botulinum; Yersinia spp., e.g. Yersinia pestis; Treponema spp.; Burkholderia spp.; e.g. Burkholderia mallei and B pseudomallei.

45. The method of claim 40, wherein the infection is a fungal infection by one or more of the following species selected from the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp., Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

46. The method of claim 45, wherein the infection is by Candida spp., (e.g. C. albicans) or Aspergillus fumigatus.

47. The method according to claim 40, wherein the disease associated with the infection is selected from the group consisting of: candidiasis (including OPC), aspergillosis (including bronchopulmonary aspergillosis, chronic pulmonary aspergillosis and aspergillomata), athlete's foot; basidiodiabolomycosis; blastomycosis; coccidioidomycosis cryptoccocis; basal meningitis; dermatophytosis; onchomycosis; dermatophytids; endothrix; exothrix; fungal meningitis, fungemia, histoplasmosis, mycosis, myrinogmycosis, paracoccidioidomycosis, penicilliosis, piedra, pneumocytosis pneumonia, sporptrichosis, tinea, zeospora and zygomycosis.

48. The method of claim 40 wherein cystamine is used in combination with an antibiotic.

49. The method of claim 48, wherein the antibiotic is selected from one or more of the group consisting of: Tobramycin, Colistin, Gentamicin or Ciprofloxacin.

50. A method of preventing biofilm formation in an environment comprising the step of administering an effective amount of cysteamine to the environment.

51. A method according to claim 50, wherein the environment comprises a biofilm forming microorganism selected from bacteria and fungi.

52. A method as claimed in claim 51 wherein the microorganism is a bacterium.

53. A method as claimed in claim 52 wherein the bacterium is selected from the group consisting of may include Pseudomonas spp., Staphylococcus spp., Haemophilus spp., Burkholderia spp., Streptococcus spp., Propionibacterium spp.

54. A method as claimed in claim 53 wherein the bacterium is selected from Pseudomonas spp., and Staphylococcus spp.

55. A method as claimed in claim 54 wherein the bacterium is Pseudomonas aeruginosa, Staphylococcus aureus or Staphylococcus epidermidis.

56. A method as claimed in claim 50 wherein the environment is the mouth.

57. A method as claimed in claim 50 wherein the microbial infection is a systemic infection.

58. A method as claimed in claim 57 wherein the systemic infection is a mucosal infection.

59. A method as claimed in claim 58 wherein the mucosal infection is a gastrointestinal, urogenital or respiratory infection.

60. A method as claimed in claim 59 wherein the mucosal infection is cystic fibrosis.
